(19) United States
(12) Patent Application Publication
(10) Pub. No.: US 2009/0143634 A1
(43) Pub. Date: Jun. 4, 2009

(54) Brachytherapy Balloon Features

(75) Inventors: Maria Benson, West Boylston, MA (US); Donna Allan, Bedford, NH (US); Walter Ocampo, Marlborough, MA (US)

Correspondence Address: CYTYC CORPORATION;Darry Pattinson, Sr. IP Paralegal, 250 CAMPUS DRIVE, MARLBOROUGH, MA, 01752, US

(73) Assignee: Cytyc Corporation, Marlborough, MA (US)

(21) Appl. No.: 11/949,882

(22) Filed: Dec. 4, 2007

(51) Int. Cl. A61N 5/00

(52) U.S. Cl. 600/3

(57) ABSTRACT

A brachytherapy treatment device includes a tubular insertion member and an expandable chamber. The tubular insertion member has a proximal end and a distal end and an expandable chamber disposed on the distal end of the tubular insertion member. The expandable chamber defines an enclosed space and has inner and outer surfaces defining a wall, wherein the wall has at least first and second wall thicknesses. The expandable chamber may comprise a balloon. A main body portion of the balloon has the first wall thickness and ribs have the second wall thickness. The ribs may be disposed to be approximately parallel or perpendicular to the tubular insertion member around the circumference of the balloon. The ribs or other thickened areas provide improved symmetry, stability, and strength to an inflated balloon. Methods of forming a symmetrical radiation dosing profile are also disclosed herein.

### TECHNICAL FIELD

This technology relates generally to brachytherapy devices and methods for use in treating proliferative tissue disorders.

### BACKGROUND

Body tissues subject to proliferative tissue disorders, such as malignant tumors, are often treated by surgical resection of the tumor to remove as much of the tumor as possible. Unfortunately, the infiltration of the tumor cells into normal tissues surrounding the tumor may limit the therapeutic value of surgical resection because the infiltration can be difficult or impossible to treat surgically. Radiation therapy may be used to supplement surgical resection by targeting the residual tumor margin after resection, with the goal of reducing its size or stabilizing it. Radiation therapy may be administered through one of several methods, or a combination of methods, such as interstitial or intercavity brachytherapy. Brachytherapy may also be administered via electronic brachytherapy using electronic sources, such as x-ray sources, for example.

Brachytherapy is radiation therapy in which the source of radiation is placed in or close to the area to be treated, such as within a cavity or void left after surgical resection of a tumor. Brachytherapy may be administered by implanting or delivering a spatially confined radioactive material to a treatment site, which may be a cavity left after surgical resection of a tumor. For example, brachytherapy may be performed by using an implantable device (e.g., catheter or applicator) to implant or deliver radiation sources directly into the tissue(s) or cavity to be treated. During brachytherapy treatment, a catheter may be inserted into the body at or near the treatment site and subsequently a radiation source may be inserted through the catheter and placed at the treatment site.

Brachytherapy is typically most appropriate where: 1) malignant tumor regrowth occurs locally, within 2 or 3 cm of the original boundary of the primary tumor site; 2) radiation therapy is a proven treatment for controlling the growth of the malignant tumor; and 3) there is a radiation dose-response relationship for the malignant tumor, but the dose that can be given safely with conventional external beam radiotherapy is limited by the tolerance of normal tissue. Interstitial and/or intercavity brachytherapy may be useful for treating malignant brain and breast tumors, among other types of proliferative tissue disorders.

There are two basic types of brachytherapy, high dose rate and low dose rate. These types of brachytherapy generally include the implantation of radioactive “seeds,” such as palladium or iodine, into the tumor, organ tissues, or cavity to be treated. Low dose rate (LDR) brachytherapy refers to placement of multiple sources (similar to seeds) in applicators or catheters, which are themselves implanted in a patient's body. These sources are left in place continuously over a treatment period of several days, after which both the sources and applicators are removed. High dose rate brachytherapy (HDR) uses catheters or applicators similar to those used for LDR. Typically, only a single radiation source is used, but of very high strength. This single source is remotely positioned within the applicators at one or more positions, for treatment times which are measured in seconds to minutes. The treatment is divided into multiple sessions (‘fractions’), which are repeated over a course of a few days. In particular, an applicator (also referred to as an applicator catheter or treatment catheter) is inserted at the treatment site so that the distal region is located at the treatment site while the proximal end of the applicator protrudes outside the body. The proximal end is connected to a transfer tube, which in turn is connected to an afterloader to create a closed transfer pathway for the radiation source to traverse. Once the closed pathway is complete, the afterloader directs its radioactive source (which is attached to the end of a wire controlled by the afterloader) through the transfer tube into the treatment applicator for a set amount of time. When the treatment is completed, the radiation source is retracted back into the afterloader, and the transfer tube is disconnected from the applicator.

A typical applicator catheter comprises a tubular member having a distal portion which is adapted to be inserted into the patient's body, and a proximal portion which extends outside of the patient. A balloon is provided on the distal portion of the tubular member which, when placed at the treatment site and inflated, causes the surrounding tissue to substantially conform to the surface of the balloon. In use, the applicator catheter is inserted into the patient's body, for instance, at the location of a surgical resection to remove a tumor. The distal portion of the tubular member and the balloon are placed at, or near, the treatment site, e.g. the resected space. The balloon is inflated, and a radiation source is placed through the tubular member to the location within the balloon.

Several brachytherapy devices are described in U.S. Provisional Patent Application 60/870,690, entitled “Brachytherapy Device and Method,” and U.S. Provisional Patent Application 60/870,670, entitled “Asymmetric Radiation Dosing for Devices and Methods,” both filed on Dec. 19, 2006, and U.S. patent application Ser. No. 11/895,559 entitled “Fluid Radiation Shield for Brachytherapy,” which are both commonly owned with the present application; U.S. Pat. No. 5,429,582; U.S. Pat. No. 5,931,774; and U.S. Pat. No. 6,482,142; each of which is hereby incorporated by reference herein in their entireties.

The dose rate at a target point exterior to a radiation source is inversely proportional to the square of the distance between the radiation source and the target point. Thus, previously described applicators, such as those described in U.S. Pat. No. 6,482,142, issued on Nov. 19, 2002, to Winkler et al., are symmetrically disposed about the axis of the tubular member so that they position the tissue surrounding the balloon at a uniform or symmetric distance from the axis of the tubular member. In this way, the radiation dose profile from a radiation source placed within the tubular member at the location of the balloon is symmetrically shaped relative to the balloon. In general, the amount of radiation desired by a treating physician is a certain minimum amount that is delivered to a region up to about two centimeters away from the wall of the excised tumor, i.e. the target treatment region. It is desirable to keep the radiation that is delivered to the tissue in this target tissue within a narrow absorbed dose range to prevent over-exposure to tissue at or near the balloon wall, while still delivering the minimum prescribed dose at the maximum prescribed distance from the balloon wall (i.e. the two centimeter thickness surrounding the wall of the excised tumor).

However, in some situations, such as a treatment site located near sensitive tissue like a patient's skin, the symmetric dosing profile may provide too much radiation to the sensitive tissue such that the tissue suffers damage or even necrosis. In such situations, the dosing profile may cause unnecessary radiation exposure to healthy tissue or it may damage sensitive tissue, or it may not even be possible to perform a conventional brachytherapy procedure. In these situations an asymmetrical dosing profile may be advantageous.

Regardless of whether a symmetric or asymmetric radiation dosing profile is desired, it is important for the balloon to be symmetrical. The inflation or deployment of a symmetrical balloon applies even pressure to surrounding tissue to symmetrically displace tissue to form a symmetrical target treatment site. Having a symmetrical target treatment site is an important preliminary consideration in performing treatment planning. Once a symmetrical target treatment site is established, then a physician can more accurately calculate the desired radiation dosing profile, which may be symmetrical or asymmetrical depending upon other considerations, such as skin spacing.

Additionally, brachytherapy treatment balloons must be stable and strong and should not weaken over time or during shelf-life of the device. Weakened areas of a balloon or uneven aging of the materials used to construct the balloon can lead to undesirable asymmetric balloon shapes upon inflation of the balloon by a physician. As a first step of a typical brachytherapy procedure, a physician is instructed to inflate or deploy the balloon and visually inspect the balloon for symmetry (as well as for product damage and cosmetic appearance). If the balloon is not symmetrical, then the device is rejected as faulty and another device is selected.

Accordingly, there remains a need for brachytherapy devices and methods having symmetrical balloon features.

### SUMMARY

Brachytherapy treatment devices and methods are disclosed herein. In one embodiment, a brachytherapy treatment device has an insertion member and an expandable chamber. The tubular insertion member has a proximal end and a distal end and an expandable chamber disposed on the distal end of the tubular insertion member. The expandable chamber defines an enclosed space and has inner and outer surfaces defining a wall, wherein the wall has at least first and second wall thicknesses. The expandable chamber may comprise a balloon. The first wall thickness is a main body portion of the balloon and the second wall thickness comprises ribs disposed on or within the balloon. The ribs may be disposed to be approximately parallel or perpendicular to the tubular insertion member. The ribs or other thickened areas provide improved symmetry, stability, and strength and form a symmetrical balloon.

The expandable chamber of the brachytherapy treatment device disclosed herein has features or thickened areas to make it stable, strong, and symmetrical. The symmetrical expandable chamber may also be oriented symmetrically relative to an inner boundary of target tissue at a treatment site. However, depending upon the positioning of the radiation source within the expandable chamber, these brachytherapy treatment devices and methods may provide either an asymmetric or symmetric radiation dosing profile relative to an inner boundary of target tissue at a treatment site.

In another embodiment, a method for creating a symmetrical radiation dosing profile at a treatment site is disclosed. The method includes: i) providing a brachytherapy treatment device comprising a tubular insertion member and an expandable chamber; the tubular insertion member has a proximal end and a distal end; the expandable chamber defines an enclosed space and is disposed on the distal end of the tubular insertion member, the expandable chamber has inner and outer surfaces defining a wall, wherein the wall has at least first and second wall thicknesses; ii) inserting the brachytherapy treatment device with the expandable chamber disposed at the treatment site; iii) deploying the expandable chamber at the treatment site, wherein the at least first and second wall thicknesses provide a symmetrically deployed expandable chamber; and iv) positing a radiation source centrally within the expandable chamber via the tubular insertion member, wherein the symmetrically deployed expandable chamber and central positioning of the radiation source provide a symmetrical radiation dosing profile at an inner boundary of the treatment site.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

FIG. 1A illustrates a side view in elevation of a first exemplary expandable chamber having ribs disposed approximately parallel to the tubular insertion member;

FIG. 1B illustrates a cross-sectional view of FIG. 1A;

FIG. 2A illustrates a side view in elevation of a second exemplary expandable chamber having ribs disposed approximately parallel to the tubular insertion member;

FIG. 2B illustrates a cross-sectional view of FIG. 2B;

FIG. 3 illustrates a side view in elevation a third exemplary expandable chamber having ribs disposed approximately perpendicular to the tubular insertion member;

FIG. 4 illustrates a cross-sectional side view of a fourth exemplary expandable chamber;

FIG. 5 illustrates a cross-sectional side view of a fifth exemplary expandable chamber;

FIG. 6 schematically illustrates exemplary expandable chamber symmetry; and

FIG. 7 is a flow diagram illustrating an exemplary operation of creating a symmetrical radiation dosing profile at a treatment site.

### DETAILED DESCRIPTION

The brachytherapy treatment devices and methods disclosed herein have expandable chambers or balloons having features or thickened portions which provide improved symmetry, stability, strength to the expandable chamber when inflated. The improvement in the symmetry, stability and strength of the expandable chamber will improve the functionality and reliability of brachytherapy devices having expandable chambers thereon. Referring now to the drawing figures, like numerals indicate like features throughout the drawing figures shown and described herein.

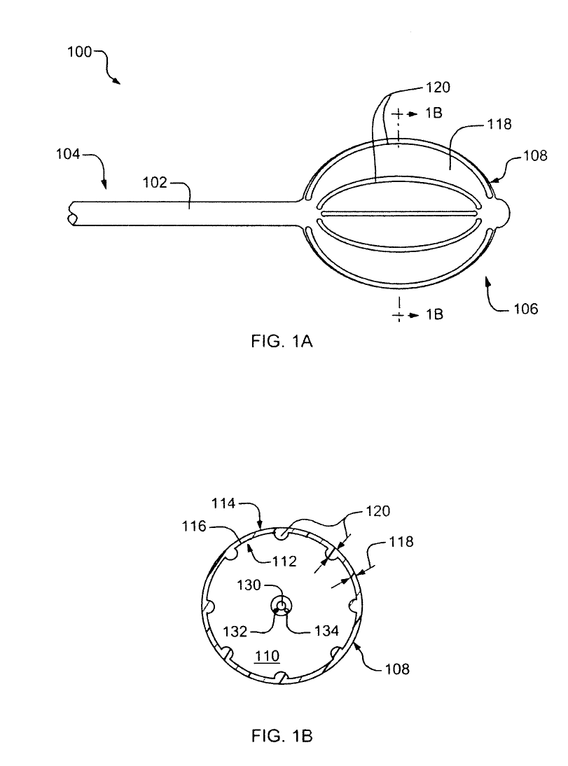

FIG. 1A illustrates a first exemplary expandable chamber and FIG. 1B illustrates a cross-sectional view of FIG. 1A. With reference to FIGS. 1A and 1B, a brachytherapy applicator or treatment device 100 (also commonly referred to as an applicator catheter or treatment catheter) may comprise an elongated tubular insertion member 102 having a proximal end 104 and a distal end 106. The distal end 106 is adapted to be inserted into a patient's body and the proximal end 104 is adapted to extend outside of the patient's body.

The insertion member 102 may be formed of a flexible material, including without limitation various plastic or elastomeric polymers and/or other suitable materials. The insertion member 102 should be flexible and soft enough that it conforms to surrounding tissue and easily bends when force is applied, such as by movement of the patient's body, making the insertion member 102 more comfortable. The insertion member 102 may further comprise a malleable element, such as a wire, adapted to confer a shape upon at least a portion of its length. The walls of the insertion member 102 may be substantially impermeable to fluids, except where there are apertures and/or openings disposed within the walls of the tubular insertion member 102.

The device 100 may further comprise an expandable chamber 108 disposed on the distal end 106 of the tubular insertion member 102. The expandable chamber 108 defines an enclosed space 110 and has inner 112 and outer 114 surfaces defining a wall 116. The wall 116 has at least first 118 and second 120 wall thicknesses, which will be described in more detail below. It should be noted that illustration of expandable chamber 108 in the attached figures is exemplary only for purposes of illustration herein and expandable chamber 108 shown in the figures may be interpreted as being in either an inflated or uninflated state.

The enclosed space 110 may be substantially or partly enclosed and defines a three-dimensional volume therein. The volume defined by the expandable chamber 108, when inflated, should be substantially similar to the volume of a lumpectomy cavity or target treatment site to substantially fill the cavity and help provide a substantially uniform and symmetrical boundary. The expandable chamber 108 may be any device which can be controllably expanded and contracted to retract surrounding tissue, such as a balloon, cage, or other device. Further, the expandable chamber 108 may be formed of a stretchy elastomeric material, such as a balloon may be made of. Alternatively, expandable chamber 108 may be formed of a more rigid or non-elastomeric material, similar to that of a bladder. Expandable chamber 108 may be inflated to elongate or expand longitudinally, as well as expanding laterally.

The expandable chamber 108 may be formed of a variety of different materials, combinations of materials, and/or blends. The expandable chamber 108 may be formed of biocompatible polymers. Some exemplary biocompatible polymers may include silastic rubbers, polyurethanes, polyethylene, polypropylene, silicone, and polyester, just to name a few examples. The wall 116 of the expandable chamber 108 may be formed of a radiation transparent material to allow radiation to pass through the wall 116 of the expandable chamber 108 to treat the tissue of the cavity surrounding the expandable chamber 108. In alternative embodiments, the wall 116 of the expandable chamber 108 may have thickened portions or features 120 which may have radiation attenuation or shielding properties. Additionally, it may be desirable to use one or more expandable chambers 108 or double-walled chambers to minimize the risk of fluid leakage from the expandable chamber 108 into a patient, such as may occur if one chamber becomes punctured.

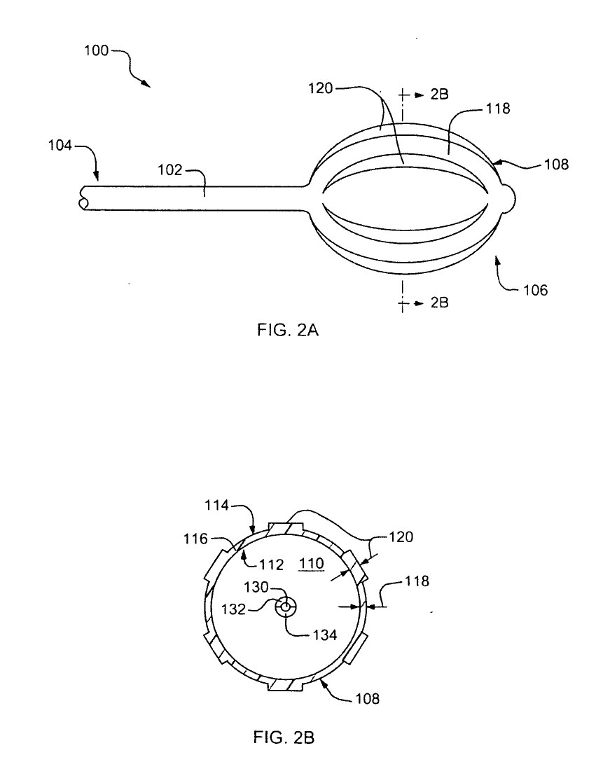

As shown in FIGS. 1B and 2B, the wall 116 may have at least first 118 and second 120 wall thicknesses. The first wall thickness 118 may be substantially uniform and comprise a main body portion of the expandable chamber 108. The second wall thickness 120 may comprises portions or areas having a thickness greater than the first wall thickness 118. The second wall thickness 120 may be features built into wall 114, such as thickened areas or ribs 120. Expandable chamber 108 may comprise a plurality of features 120 having a second wall thickness 120, which may have any number of different geometries, such as differing shapes, sizes, widths, and/or lengths.

Features 120 may be formed of a variety of different materials or combinations of materials. Additionally, the features 120 may be formed to have the same or different properties from that of the first wall thickness 118. In one embodiment, features 120 may be formed as a ribbon of material built into wall 114, such as a rib. In some exemplary implementations, features 120 may have various different thicknesses and may have a thickness only minimally greater than that of first wall thickness 118. Additionally, device 100 may comprise more than first 118 and second 120 wall thicknesses, and thus may have third, fourth, fifth, etc. areas having different wall thicknesses. Device 100 may also have areas of continually varying wall thicknesses 120, such as areas having differing depth, width, and breadth.

Features 120 may be formed within expandable chamber 108 or disposed on an inner 112 or outer 114 surface of the expandable chamber wall 114 using a variety of different techniques. In some exemplary embodiments, features 120 may be formed in expandable chamber 108 wall 116 using blow molding techniques, extrusion, liquid injection molding, or dip molding for example. In some implementations, such as when expandable chamber 108 is formed of polyurethane, a combination of extrusion and blow molding techniques may be utilized to form features 120. In other implementations, such as when expandable chamber 108 is formed of silicone, features 120 may be directly molded in, such as by liquid injection molding may be utilized to form features 120. When expandable chamber 108 is in a fully inflated state, some of the ratios between features 120 may be slightly altered, as will be known by one of ordinary skill in the art after having become familiar with the teachings herein.

Features 120 may help strengthen expandable chamber 108, by reducing or eliminating expandable chamber 108 burst during use of the device 100. The features 120 may be used to increase strength and stability of the inflated shape of the expandable chamber 108. Additionally, features 120 help to ensure a more symmetrical or uniform shape of inflated expandable chamber 108, which increases stability and shelf-life of the device 100. The stabilization results from the increased thickness of features 120, which are more resistant to deformation from inflation pressure. The thickened features 120 also balance or equalize the expansion and/or elongation of the expandable chamber 108 in its relatively thinner sections 118 (i.e., first wall thickness 118).

As shown in FIGS. 1A, 1B, 2A, and 2B, the features 120 may comprise a plurality of ribs 120. The plurality of ribs 120 may comprise any number of ribs formed of a variety of different lengths and widths and the number of ribs 120 shown in FIGS. 1A and 2A are exemplary only for purposes of illustration herein. Ribs 120 may be formed integrally within wall 116 of expandable chamber 108 and may extend inward of inner surface 112 (as shown in FIG. 1B) or may extend outward of outer surface 114 (as shown in FIG. 2B). As shown in FIGS. 1A and 1B, the ribs 120 may comprise a plurality of tall, skinny, and approximately half-circular shaped ribs disposed radially around the circumference of the expandable chamber 108. In this embodiment, the ribs 120 have a height substantially greater than their width. The ribs 120 may be disposed to be approximately parallel to tubular insertion member 102 and main lumen 130, as shown in FIG. 1A. As shown in FIG. 1B, the ribs 120 may be formed within wall 116 so that they protrude inward of inner surface 112 toward main lumen 130.

As shown in FIGS. 2A and 2B, the ribs 120 may comprise a plurality of wide, flat, and approximately rectangular shaped ribs disposed radially around the circumference of the expandable chamber 108. In this embodiment, the ribs 120 have a width substantially greater than their height. The plurality of ribs 120 may be radially disposed around the circumference of the expandable chamber 108. Further, the plurality of ribs 120 may be positioned approximately parallel to tubular insertion member 102 and main lumen 130, as shown in FIG. 2A. As shown in FIG. 2B, the ribs 120 may be formed within wall 116 so that they protrude outward of outer surface 114 away from enclosed space 110 and main lumen 130.

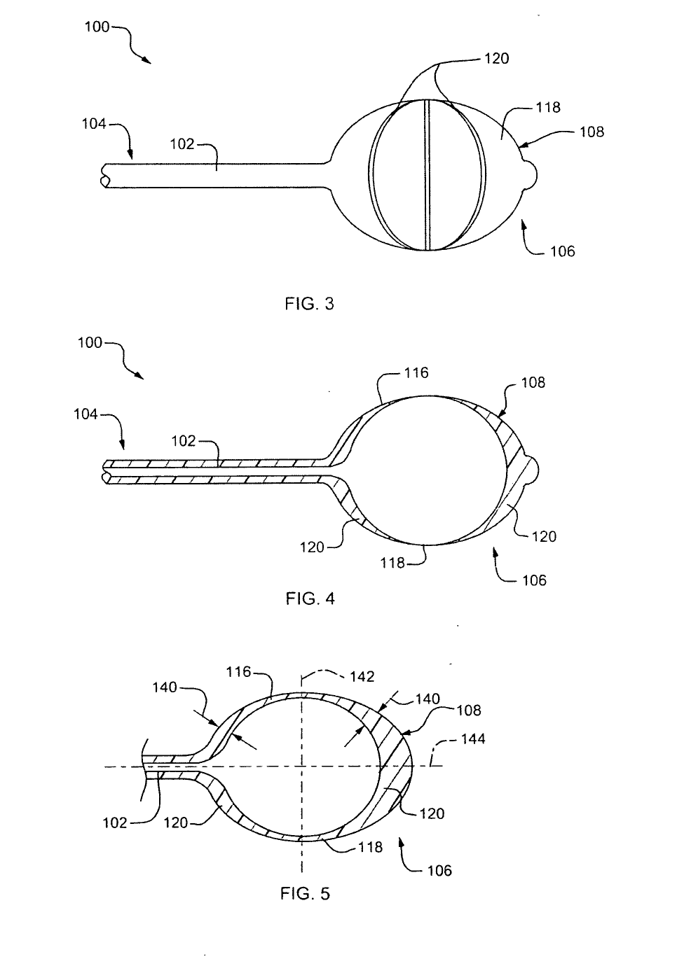

With reference now to FIG. 3, a plurality of ribs 120 may also be radially disposed around the circumference of the expandable chamber 108. Further, the plurality of ribs 120 may be positioned approximately perpendicular to tubular insertion member 102 and main lumen 130. Any number of ribs 120 may be utilized and FIG. 3 illustrates three ribs on one-half of expandable chamber only for exemplary purposes of illustration herein.

In another embodiment shown in cross-section in FIG. 4, features 120 may comprise two portions (shown as 120) disposed on proximal 104 and distal 106 ends of the expandable chamber 108 at positions most adjacent to and circumferentially around the tubular insertion member 102. The features 120 comprise the portions of second wall thickness 120, while the remaining main body portion of the wall 116 of expandable chamber 108 may be formed having first thickness 118. The features 120 may have a maximum thickness where the expandable chamber 108 is coupled to insertion member 102. Features 120 within wall 116 may taper or gradually thin in correlation with increasing distance from the point where the expandable chamber 108 is coupled to the insertion member 102, as shown in FIG. 4. The positioning of the features 120 adjacent to the tubular insertion member 102 compensates and provides additional support for the thinner areas (having first thickness 118) to improve stability, strength, and symmetry of the inflated expandable chamber 108.

As shown in FIG. 5, the wall thickness 120 may vary throughout the wall 116 of the expandable chamber 108. In this embodiment, the features 120 comprise the portions of second wall thickness 120, while the remaining main body portion of the wall 116 of expandable chamber 108 may be formed having first thickness 118. The wall 116 may have a first maximum thickness 140 at a distal portion of the expandable chamber 108 and taper to a first minimal thickness at a position 90° radially (shown as 142) from a center axis 144 of the tubular insertion member 102. Additionally, the wall 116 has a second maximum thickness at a proximal portion of the expandable chamber and tapers to a second minimal thickness at a position 90° radially (shown as 142) from a center axis 144 of the tubular insertion member. Said another way, the expandable chamber 108 may have a maximum wall thickness at its minimal points of inflation, and minimum wall thickness at its maximal points of inflation.

Similar to FIG. 4, the positioning of the features 120 adjacent to the tubular insertion member 102 compensates for and provides additional support for the thinner areas (having first thickness 118) to improve stability, strength, and symmetry of the inflated expandable chamber 108. In alternative embodiments, the areas, positions, and arrangements of first wall thickness 118 and second wall thickness 120 may be changed. In other embodiments, the exact positioning of the features 120 and the locations where the features 120 begin and end may also be altered.

As shown in FIGS. 1B and 2B, the elongated tubular insertion member 102 may also include a main lumen 130 extending between and operably coupling the proximal 104 and distal 106 ends of the tubular insertion member 102. The main lumen 130 may be a radiation source pathway configured to receive a radiation source and provide a pathway for positioning a radiation source at radiation source position located approximately centrally within main lumen 130 within the expandable chamber 108. In alternative embodiments, there may be multiple source lumens configured to receive a radiation source and provide pathways for positioning a radiation source at similar or different positions within the expandable chamber 108.

The main lumen 130 of the insertion member 102 may further comprise a plurality of other tubes or lumens 132, 134 disposed therein to provide several separate and independently operable pathways for accessing the distal end 106 of the insertion member 102 via the proximal end 104 of the insertion member 102. These secondary lumens 132, 134 may be offset from the approximately central position of the main lumen 130 and may be used for injection and evacuation of fluids into enclosed spaced 110 defined by wall 116 of expandable chamber 108. As shown variously in FIGS. 1B and 2B, the shapes, sizes, and arrangement of these secondary lumens 132, 134 may be varied. Curving, bending or articulating of the lumens 130, 132, 134 may provide multiple alternative radiation source positions within the expandable chamber, thus providing multiple options for asymmetric orientation of the isodose profile and for treatment planning.

An exemplary brachytherapy treatment device 100 may also have a hub (not shown) disposed on the proximal end 104 of the insertion member. The hub may have one or a plurality of ports (not shown) operably coupled to main lumen 130 and/or secondary lumens 132, 134. The plurality of ports on the hub are configured to remain outside of the patient's body while being operably coupled to the distal end 106 of the device 100. The plurality of ports are configured to allow a physician access to the distal end 106 of the device 100, such as by inflation or evacuation of fluids into/out of expandable chamber 108. One of the ports, such as a port coupled to main lumen 130, may be configured to receive a radiation source. The ports may be formed of appropriate materials, such as plastic for example, and may be sealed to prevent leakage of fluids from the main lumen 130 and/or secondary lumens 132, 134.

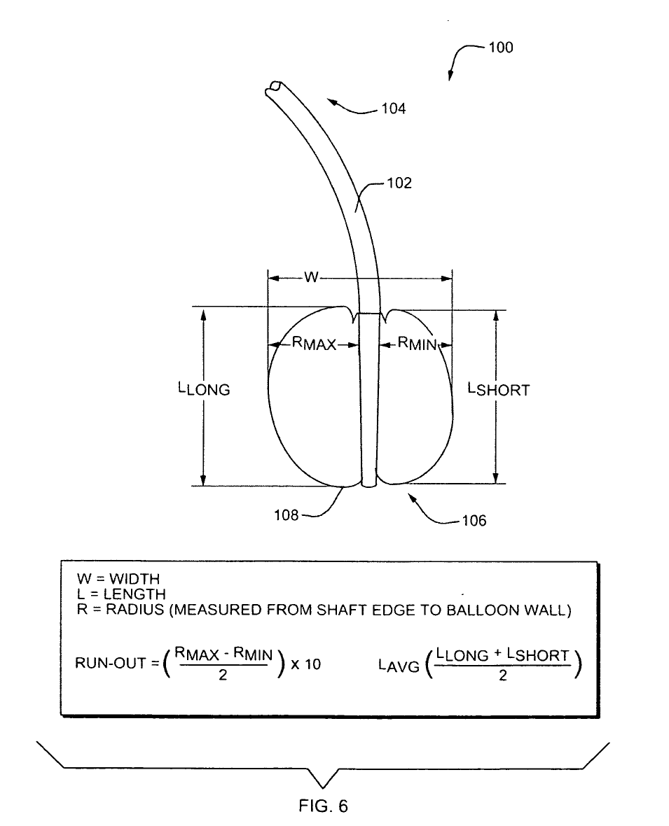

The brachytherapy treatment devices 100 disclosed herein provide a symmetrical expandable chamber 108 or balloon to enhance treatment planning and functionality of the brachytherapy device 100. FIG. 6 schematically illustrates expandable chamber 108 symmetry calculations. As shown in FIG. 6, various different dimensions (e.g., width, length, and radius) of an expandable chamber 108 will be determined and plugged into a formula to determine runout. If the resulting runout value exceeds a predetermined maximum value, then the expandable chamber 108 will be declared asymmetrical or defective. If the resulting runout value is less than a predetermined maximum value, then the expandable chamber 108 is determined to be within design tolerances and symmetrical.

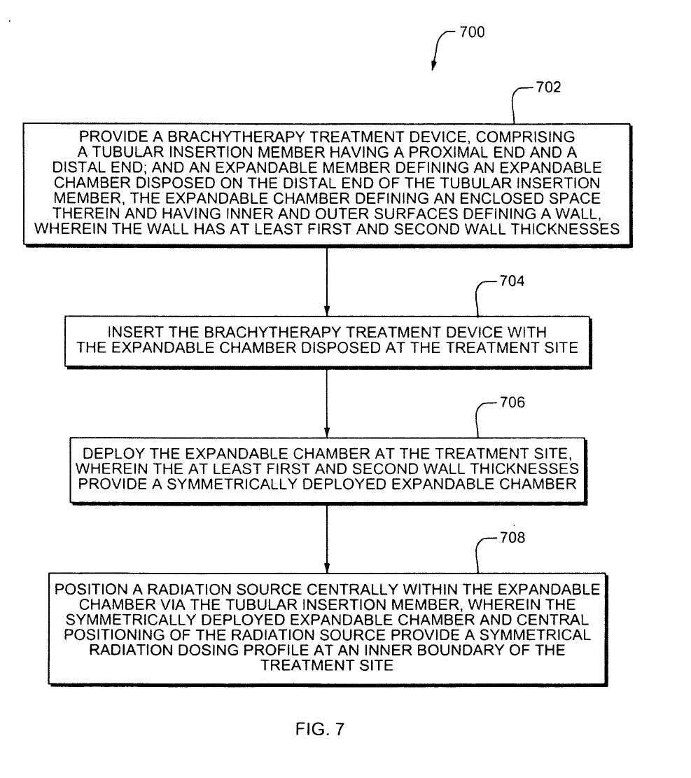

Methods for delivering brachytherapy treatment to a target treatment site in a patient are also provided herein. One exemplary method 700 for creating a symmetric radiation dosing profile at a treatment site is shown generally in FIG. 7. As discussed above, the symmetrical expandable chambers 108 disclosed herein may also be used in combination with an off-set radiation source position to create an asymmetric dosing profile.

The method of creating a symmetric radiation dosing profile begins by providing 702 a brachytherapy treatment device 100 comprising a tubular insertion member 102 and an expandable chamber 108. As described in detail above, the tubular insertion member 102 has a proximal end 104 and a distal end 106: the expandable chamber 108 defines an enclosed space and is disposed on the distal end 106 of the tubular insertion member 102. The expandable chamber 108 has inner 112 and outer 114 surfaces defining a wall 116, wherein the wall 116 has at least first 118 and second 120 wall thicknesses.

The method 700 continues by inserting 704 the brachytherapy treatment device 100 with the expandable chamber 108 disposed at the treatment site. Prior to inserting 704 or placing of the device 100, it is common for a surgery or lumpectomy to have been performed to remove as much of a tumor as possible. A surgical resection of the tumor is typically performed, leaving a resected space or cavity for placement of the catheter within the patient. In some embodiments, the placement of the catheter may be done using a previously made incision (such as that used for the lumptectomy) or may include formation of a new or different incision.

The expandable chamber 108 is then deployed 706 at the treatment site. The at least first 118 and second 120 wall thicknesses provide a symmetrically deployed or inflated expandable member 108. The expandable chamber 108 may be inflated (e.g., by injection of fluid), for example, to fill the cavity of a resected tumor. The target tissue surrounding the cavity may substantially conform to the outer surface 114 or wall 116 of the expandable chamber 108. In this manner, the tissue surrounding the cavity may also be positioned to reshape tissue to provide a symmetrically shaped cavity. This symmetrically shaped cavity is an important factor in the calculation of the treatment plan for the patient.

After deploying 706 the expandable chamber 108, a radiation source is then positioned 708 centrally within the expandable chamber 108 via the tubular insertion member 102. The symmetrically deployed expandable chamber 108 and central positioning of the radiation source provide a symmetrical radiation dosing profile at an inner boundary of the treatment site. Following radiation treatment, the catheter 100 may remain within the patient's body in the treatment position so that it can be used during the next treatment session, or it may be removed.

Once placed at the treatment site, the radiation source creates a radiation dose distribution profile which takes the shape of spherical isodose shells that are centered on the location of the radiation source. A target treatment site is typically an approximately circular area surrounding an inner boundary or margin of a cavity left after tumor resection. A radiation source positioned at the radiation source position will emit radiation to produce an isodose profile relative to the inner boundary of target tissue to be treated, without the effect of any radiation shielding.

The radiation dose from a radiation source is typically emitted substantially equally in all 360° surrounding the radiation source position (referred to generally as radiation dose profile), assuming the radiation source has no abnormalities or shielding thereon. Because the radiation dose is emitted substantially equally in all directions, and because it decreases based upon the square of the distance, the proximity of sensitive tissues to a radiation source will result in the sensitive tissue receiving an undesirably high and potentially very damaging dose of radiation. Thus, in some situations, it may be desirable to create an asymmetric radiation dosing profile. However, when the target treatment site is not located proximally to any sensitive tissues, a symmetrical radiation dosing profile may be desired.

Disclosed herein are devices and methods for use in treating proliferative tissue disorders by the application of radiation, energy, or other therapeutic rays. While the devices and methods disclosed herein are particularly useful in treating various cancers and luminal strictures, and a person of ordinary skill in the art will appreciate that the methods and devices disclosed herein can have a variety of configurations, they can be adapted for use in a variety of medical procedures requiring treatment using sources of radioactive or other therapeutic energy. These sources can be radiation sources such as radio-isotopes, or man-made radiation sources such as x-ray generators. The source of therapeutic energy can also include sources of thermal, radio frequency, ultrasonic, electromagnetic, and other types of energy.

It should be understood that various changes and modifications to the above-described embodiments will be apparent to those skilled in the art. The examples given herein are not meant to be limiting, but rather are exemplary of the modifications that can be made without departing from the spirit and scope of the described embodiments and without diminishing its attendant advantages.

What is claimed is:

1. A brachytherapy treatment device, comprising:
a tubular insertion member having a proximal end and a distal end; and
an expandable chamber disposed on the distal end of the tubular insertion member, the expandable chamber defining an enclosed space therein and having inner and outer surfaces defining a wall, wherein the wall has at least first and second wall thicknesses.

2. The device of claim 1, wherein the expandable chamber comprises a balloon.

3. The device of claim 1, wherein the expandable chamber is elastomeric.

4. The device of claim 1, wherein the expandable chamber is non-elastomeric.

5. The device of claim 1, wherein the first wall thickness comprises a main body portion of the expandable chamber and wherein the second wall thickness comprises ribs.

6. The device of claim 5, wherein the ribs provide symmetry to form a symmetrical expandable chamber.

7. The device of claim 5, wherein the ribs provide stability to form a stable expandable chamber.

8. The device of claim 5, wherein the ribs are formed on the inner surface of the wall.

9. The device of claim 5, wherein the ribs are formed on the outer surface of the wall.

10. The device of claim 5, wherein the ribs are have a height substantially greater than a width.

11. The device of claim 5, wherein the ribs have a width substantially greater than a height.

12. The device of claim 5, wherein the ribs are disposed approximately parallel to the tubular insertion member.

13. The device of claim 5, wherein the ribs are disposed approximately perpendicular to the tubular insertion member.

14. The device of claim 1, wherein the first wall thickness is substantially uniform throughout the expandable chamber and wherein the second wall thickness varies throughout the expandable chamber, the second wall thicknesses forming a plurality of ribs.

15. The device of claim 1, wherein the second wall thickness is disposed in two portions on ends of the expandable chamber formed adjacent to and circumferentially around the tubular insertion member.

16. The device of claim 1, wherein the wall has a first maximum thickness at a distal portion of the expandable chamber and tapers to a first minimal thickness at a position 90° radially from a center axis of the tubular insertion member and wherein the wall has a second maximum thickness at a proximal portion of the expandable chamber and tapes to a second minimal thickness at a position 90° radially from a center axis of the tubular insertion member.

17. The device of claim 1, wherein at least one of the first or second thicknesses have radiation shielding or attenuating properties.

18. A method for creating a symmetrical radiation dosing profile at a treatment site, comprising:
providing a brachytherapy treatment device, comprising:
a tubular insertion member having a proximal end and a distal end; and
an expandable chamber defining disposed on the distal end of the tubular insertion member, the expandable chamber defining an enclosed space therein and having inner and outer surfaces defining a wall, wherein the wall has at least first and second wall thicknesses;
inserting the brachytherapy treatment device with the expandable chamber disposed at the treatment site;
deploying the expandable chamber at the treatment site, wherein the at least first and second wall thicknesses provide a symmetrically deployed expandable chamber;
positing a radiation source centrally within the expandable chamber via the tubular insertion member, wherein the symmetrically deployed expandable chamber and central positioning of the radiation source provide a symmetrical radiation dosing profile at an inner boundary of the treatment site.
